(19) United States
(12) Patent Application Publication
(10) Pub. No.: US 2012/0316632 A1
(43) Pub. Date: Dec. 13, 2012

(54) Retrievable covered stent for bifurcation aneurysms

(76) Inventor: Bulang Gao, Lynn, MA (US)

(21) Appl. No.: 13/158,518

(22) Filed: Jun. 13, 2011

(51) Int. Cl. A61F 2/82

(52) U.S. Cl. 623/1.2

(57) ABSTRACT

A retrievable covered stent has a self-expandable covered stent on the distal end and is particularly useful for occluding the neck of an aneurysm located in the vicinity of bifurcations or trifurcations of vessels. The said retrievable covered stent comprises a proximal retrieving structure and a proximal self-expandable supporting stent for supporting and retaining a smaller self-expandable covered stent on the distal end which is used to seal the neck of a bifurcation aneurysm.

### FIELD OF THE INVENTION

The present invention relates to medical devices used to treat aneurysms within diseased blood vessels, and more particularly, relates to medical devices used to seal the neck of terminal aneurysms which occur in the vicinity of bifurcations or trifurcations of blood vessel. Examples of blood vessels in which the said retrievable covered stent may be implanted include the basilar artery bifurcation, the internal carotid artery bifurcation, the middle cerebral artery bifurcation or trifurcation, the anterior communicating artery bifurcation, bifurcations formed by a branch and a major artery, and other similar arterial bifurcation system.

### BACKGROUND OF THE INVENTION

Although the following discussion focuses on the treatment of vascular diseases, it is equally applicable to diseases in other locations or tracts. Although many types of vascular diseases can be treated with this retrievable covered stent such as aneurysms, arteriovenous fistulas, vascular defects and stenosis, the following discussion focuses on the treatment of intracranial bifurcation aneurysms which are one very significant use for the retrievable covered stent.

An aneurysm is a sac formed by localized dilatation of the wall of a vessel. Common areas where aneurysms occur and cause potential medical conditions include the coronary arteries, the carotid arteries, various cerebral arteries and the abdominal aorta. The wall of an aneurysm may progressively dilates, weakens and ruptures, causing dramatic negative health consequences such as a stroke or death when a cerebral aneurysm or an abdominal aortic aneurysm ruptures. Aneurysms can be treated surgically or endovascularly. The surgical procedure, however, is extremely traumatic and presents a high level of risk, particularly when treating cerebral aneurysms. To avoid the high risk of vascular surgery, endovascular devices have been used to either cover or fill an aneurysm with embolic materials like metallic coils. One method of endovascular treatment is to fill the aneurysm cavity with coils alone or with coils. When using coils in combination with stents (this technique is also called stent-assisted coil embolization), one or two non-covered stents have to be placed across the aneurysm neck in advance before coil embolization is performed through the stent struts into the aneurysm cavity. Once the aneurysm cavity is filled with coils, blood will not enter the aneurysm, and the aneurysm will be expelled from blood circulation. However, for large aneurysms, a lot of coils have to be used, thus creating a mass effect with severe consequences. Moreover, a lot of other severe complications may also occur in coiling embolization such as coil protrusion or escaping, intra-procedural aneurysm rupture leading to subarachnoid hemorrhage, and thrombosis caused by coils leading to cerebral embolism. These complications limit its application. Another infrequently-used method is the deployment of a covered stent across the aneurysm neck, and this approach is a much better method than embolization of the aneurysm with embolic materials such as coils alone or coils combined with stents. The covered stent's membrane or graft can act as a barricade to prevent blood from entering the aneurysm sac, leading to thrombosis within the sac, consequently expelling the aneurysm from blood circulation. At the same time, the parent artery harboring the aneurysm is protected. Since manipulation of the covered stent occurs within the parent artery rather than within the aneurysm cavity as in coiling, there is no possibility of intra-procedural aneurysmal rupture. Furthermore, the thrombus formed within the aneurysm sac will not be dislodged by the covered stent, which greatly decreases the possibility of thromboembolic complications. For patients with very large or giant aneurysms where embolization could require the placement of many costly coils which may result in a mass effect, the deployment of a covered stent over the aneurysm orifice may sufficiently occlude the aneurysm without causing those side effects mentioned above. Moreover, small and recurrent aneurysms difficult to treat can also be easily completely occluded by a covered stent. However, the current covered stents available are only good for aneurysms on straight vessels without branches involved and can not be used for aneurysms at vascular bifurcations or trifurcations.

Aneurysms at vascular bifurcations like the basilar artery bifurcation, the internal carotid artery bifurcation and the middle cerebral artery bifurcation usually have large aneurysmal necks which make regular endovascular embolization with coiling alone or stent-assisted coiling (non-covered stents) extremely difficult. Currently, because there are no suitable covered stents for such large aneurysms at vascular bifurcations or trifurcations, the endovascular management of these large-neck bifurcation aneurysms is by using stent-assisted coiling (or coils in combination with sents). After one or two non-covered stents have been placed across the aneurysm neck, coiling is performed through the stent struts into the aneurysm cavity. This type of endovascular treatment is very complex and complicated with a high rate of complications including stent migration, coil protrusion into the artery lumen, aneurysm perforation leading to re-bleeding, embolus formation and subsequent embolism, artery injury, and so on and so forth.

Currently, the stent used in the endovascular treatment of vascular diseases in the intracranial vasculature is not retrievable especially for covered stents. This means the stent will remain in the same position and state once it has been deployed. It can not be retrieved, readjusted and redeployed even though it is not in the desirable place without complete occlusion of the aneurysm and may damage the blood vessels. For a retrievable stent, it can be retrieved, readjusted and redeployed to the most desirable position, and in this way, it will greatly benefit the endovascular treatment of vascular diseases.

Therefore, there is a need for the invention of a retrievable covered stent which can easily be used to treat bifurcation aneurysms and be retrieved and redeployed most appropriately if it is not deployed in the desirable place.

### BRIEF SUMMARY OF THE INVENTION

The present invention is related to a retrievable covered stent which is particularly useful for treating intracranial aneurysms at the bifurcation or trifurcation of blood vessels. The retrievable covered stent has a proximal supporting stent for supporting and retaining a smaller self-expanding covered stent on the distal end of the proximal supporting stent. The working mechanism of the retrievable self-expandable covered stent in treating bifurcation aneurysms is through expanding the smaller distal self-expandable covered stent to occlude the neck of the aneurysm at the vascular bifurcation. Once the neck of the aneurysm is occluded by the distal covered stent, blood flow will not be able to get into the aneurysm cavity, and clot will be formed within the aneurysm, leading to complete elimination of the aneurysm from blood circulation and consequent heal of the aneurysm. If the retrievable covered stent is not deployed in the best location, the stent can be retrieved, readjusted and redeployed in the best position for treating the bifurcation aneurysm. Examples of bifurcations where the retrievable covered stent may be implanted include, without limitation: the basilar artery bifurcation, the middle cerebral bifurcation or trifurcation, the internal carotid artery bifurcation, the anterior communicating bifurcation, bifurcations formed by a branch and a major artery, and other similar arterial bifurcation system.

The said retrievable covered stent comprises a proximal retrieving structure, a tubular supporting stent and a distal smaller self-expandable covered stent which is consisted of a smaller self-expandable stent and graft material covering the internal surface of the smaller self-expandable stent. The retrieving structure is used to retrieve the whole stent. The smaller self-expandable covered stent is attached to the distal end of the proximal supporting stent and is mainly used to seal the neck of an aneurysm at vascular bifurcations.

### BRIEF DESCRIPTION OF THE DRAWINGS OF THE INVENTION

Examples of embodiments of the invention will now be described with reference to the following drawings.

FIG. 1 shows different types of the retrievable self-expandable covered stent with different sizes of the small moveable self-expandable covered stent (101) on the distal end. The stents are fully expanded. 101 indicates the small distal self-expandable covered stent in the expanding state like an umbrella which can be used to seal the aneurysm neck. 105 indicates the proximal retrieving structure used for retrieving the whole stent.

FIG. 2 demonstrates a fully expanded retrievable self-expandable covered stent with two markers (102) at the proximal end and some markers (103) at the distal end. 104 represents the proximal supporting stent for the whole retrievable self-expandable covered stent while 105 indicates the proximal retrieving structure used for retrieving the whole stent.

FIG. 3 reveals the top view of the retrievable covered stent in full expansion viewed from the distal smaller self-expandable covered stent (101) with different sizes and shapes. 113 indicates a small hole in the center of the covering membrane, and 114 is the distal end of the proximal supporting stent viewed from the distal covered stent. The small hole is for the passage of a guidewire through the covered stent.

FIG. 4 shows the retrievable self-expandable covered stent with different lengths in the compressed state (non-expanding). 101 indicates the small distal self-expandable covered stent in the compressed state, 104 refers to the proximal supporting self-expandable stent in the compressed state, and 105 is the proximal retrieving structure used for retrieving the whole stent.

FIG. 5 indicates the proximal supporting stent (104) with removal of the small distal self-expandable covered stent. 102 refers to the markers at the proximal end of the stent while 103 indicates the markers at the distal end.

FIG. 6 demonstrates the top view of the small distal moveable self-expandable covered stent (101) with different sizes and shapes (A and C). Fig. B and D indicate the corresponding self-expandable stents without the covering membrane on them. 113 indicates the small hole in the center of the covering membrane of the distal covered stent. The small hole is for the passage of a guidewire through the covered stent.

FIG. 7 shows the lateral view of the small distal self-expandable covered stent (101) with different sizes and shapes. From the lateral view, it can be seen that the distal self-expandable covered stent is like an umbrella in the expanding state.

FIG. 8 demonstrates the proximal retrieving structure (105) of the proximal supporting stent (104) for the whole retrievable self-expandable covered stent. The proximal retrieving structure (105) is a particular structure which comprises a flexible circular metallic wire (111) with two markers (102) on it (A) or on the nearby most prominent stent struts (B, 112). 112 indicates the struts of the proximal supporting stent (104). Note, the ends of the struts (112) of the proximal retrieving structure are not on the same horizontal level but on a slope. This particular structure enables the whole self-expandable covered stent to be retrievable.

FIG. 9 is the proximal supporting stent combined with the distal small self-expandable stent without the covering membrane. 102 indicates the markers at the proximal end of the stent, 103 the markers at the distal end of the stent, 104 the proximal supporting stent, and 105 the proximal retrieving structure for retrieving the stent.

FIG. 10 shows a bifurcation aneurysm with the bifurcation angle A being smaller of 115.53° and blood flow (arrows) in the parent artery, bifurcated branches and the aneurysm cavity. The small arrow indicates the aneurysm.

FIG. 11 demonstrates the stents (A and B) used for treating aneurysms at a vascular bifurcation with smaller bifurcation angles like the one shown in FIG. 10. For smaller bifurcation angles, the stents used should have a deeper and longer distal moveable self-expandable covered stent (A and B). Fig. C reveals that the appropriate deployment of the covered stent immediately occludes the aneurysm neck and blood flow can not get into the aneurysm cavity.

FIG. 12 reveals another kind of bifurcation aneurysm with the bifurcation angle A being bigger of 155.30° and blood flow (arrows) in the parent artery, bifurcated branches and the aneurysm cavity. The small arrow indicates the bifurcation aneurysm.

FIG. 13 demonstrates the stents (A and B) used for treating aneurysms at bifurcations with bigger bifurcation angles like the one shown in FIG. 12. For bigger bifurcation angles, the stents used should have a shallower and shorter distal moveable self-expandable covered stent (A and B). Fig. C reveals that the appropriate deployment of the covered stent immediately occludes the aneurysm neck and eliminates blood flow from getting into the aneurysm cavity.

FIG. 14 shows the delivery and deployment process of the whole retrievable self-expandable covered stent. 106 indicates the guidewire, 107 means the pushing structure for the deployment of the whole stent, 108 is the catheter used to deliver the stent, and 109 indicates the whole stent within the catheter.

FIG. 15 illustrates the retrieving process of the deployed retrievable self-expandable covered stent. 108 indicates the catheter, 109 is the whole stent and 110 represents the retrieving hook.

### DETAILED DESCRIPTION OF THE DRAWINGS OF THE INVENTION

Although the following discussion focuses on the treatment of vascular diseases, it is equally applicable to diseases in other locations or tracts. Although many types of vascular diseases can be treated with this retrievable self-expandable covered stent such as aneurysms, vascular defects and stenosis, the following discussion focuses on the treatment of intracranial aneurysms at vascular bifurcations.

While this invention may be embodied in many different forms, there are described in detail herein specific embodiments of the invention. This description is an exemplification of the principles of the invention and is not intended to limit the invention to the particular embodiments illustrated.

As indicated above, the present invention is directed to a variety of embodiments.

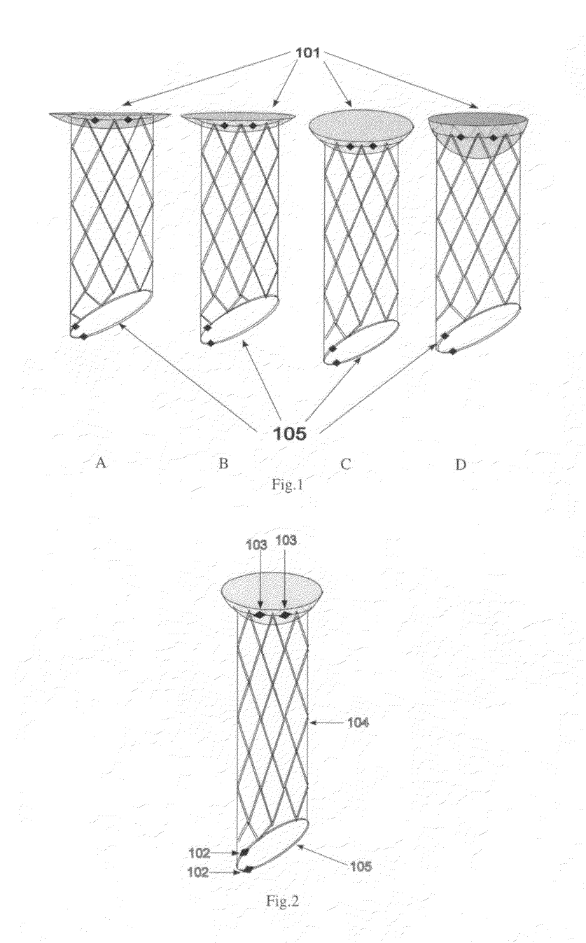

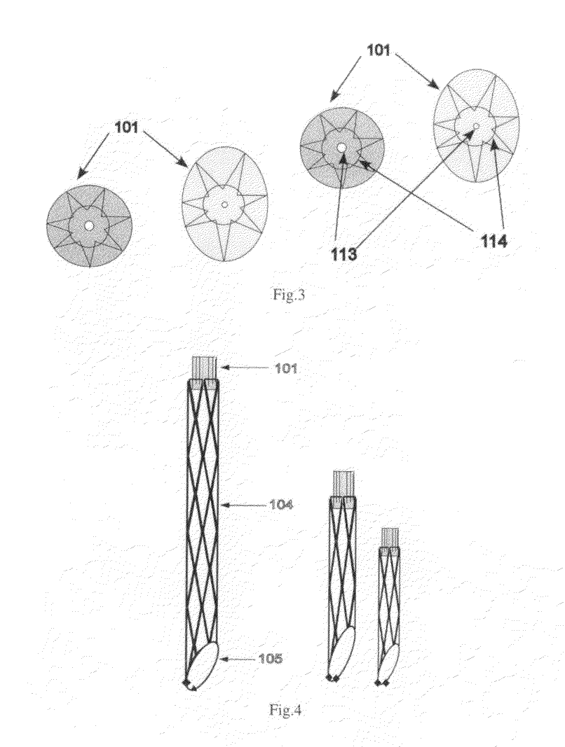

Illustrated in FIG. 1 is an exemplary design of the retrievable self-expandable covered stent with different sizes and shapes of the distal moveable self-expandable covered stent (101). The stents are fully expanded. The proximal retrieving structure (105) can be used to retrieve the whole stent. FIG. 2 is a fully expanded retrievable self-expandable covered stent with two markers (102) at the proximal end and some markers (103) at the distal end. These markers help visualization of the stent during the deployment process. The two proximal markers (102) help decide the location of retrieval at the proximal retrieving structure (105) for retrieval of the stent in case it is not deployed in the desirable location. The distal markers (103) at the distal end of the whole stent are useful for exact localization of the distal end in the blood vessels so that the bifurcation aneurysm neck can be completely occluded. If the whole stent is not properly deployed in the desirable location, a hook can be used to catch the proximal retrieving structure (105) between the two proximal markers (102) to retrieve and readjust the whole stent before re-deployment again.

When the stent is in full expansion, FIG. 3 reveals the top view of the retrievable self-expandable covered stent viewed from the distal smaller self-expandable covered stent (101) with different sizes and shapes. Different sizes and shapes of the small distal covered stent (101) can be used to occlude the aneurysm necks with different sizes and shapes at bifurcations. Based on the sizes and shapes of the neck of the bifurcating aneurysms, the retrievable covered stents with different sizes and shapes of the distal small covered stent (101) can be chosen for better occlusion effect. 113 indicates a small hole in the center of the covering membrane of the distal covered stent, and this hole allows the passage of a guidewire for navigation of the whole stent to the appropriate position. This hole is very small and will not affect the aneurysm occlusion status when the aneurysm is occluded by the distal self-expandable covered stent. The distal end (114) of the proximal supporting stent is connected to the struts of the distal self-expandable covered stent for support. The connection is from the end of the proximal supporting stent to the middle part of the stent struts of the distal self-expandable stent, and this connection is to make the distal self-expandable stent moveable.

With reference to FIG. 4, the retrievable self-expandable covered stent is in the compressed state with different lengths of the proximal supporting stent (104). The distal self-expandable covered stent (101) can, when deployed, expand into an umbrella-like structure like the one (101) shown in FIGS. 1 and 2. The proximal supporting stent (104) may adopt different lengths in order to obtain different strengths of apposition onto the vascular wall. Longer stents have stronger supporting effects. The length of the proximal supporting stent (104) is based on the length of the parent artery before bifurcating. The proximal retrieving structure (105) forms a profile smaller than the other more distal segments of the stent in the compressed state, and this small profile facilitates retrieving of the whole stent when the proximal retrieving structure (105) is pulled into a catheter for retrieving.

With reference to FIG. 5, the proximal supporting self-expandable stent (104) is in full expansion with markers (102 and 103) at both ends of the stent. The distal end of the stent struts of the supporting expandable stent is on a horizontal level while the proximal end of the stent struts of the supporting stent forms an incline plane or a slope rather than a horizontal level. The proximal slope profile of the struts of the supporting stent is used for retrieving the whole stent. There are some markers on the most prominent proximal struts or on the circular metallic wire nearby for marking the hooking position. When the whole stent is in the compressed state, the proximal end of the stent will form a smaller profile (as demonstrated in FIG. 4) than the distal stent segments so that the smaller proximal end will be easily pulled into a catheter followed by distal segments with bigger profiles. The proximal supporting stent used in this retrievable self-expandable covered stent disclosed herein may be manufactured using any suitable known techniques to make a metal sheet into a stent or to braid or weld metallic wires into a stent. The material used for the supporting stent can be of any type including steel and nitinol. The proximal supporting stent can be closed-cell design or open-cell design, and it can be self-expandable or balloon-expandable stent.

Referring to FIG. 6, the small distal self-expandable covered stent (101) is shown in Fig. A and C. Fig. B and D demonstrate the corresponding small self-expandable stent with the removal of the covering membrane. The covering membrane or graft can be of any type including biomedical materials like polytetrafluoroethylene and other macromolecular materials. The graft or membrane can be stitched or glued onto the internal surface of the stent. The membrane used to cover the stent in this invention includes suitable polymer materials like polycarboxylic acids, cellulosic polymers, nylon, collagen, polytetrafluoroethylene (PTFE) and expandable PTFE, polyethylene terephthalate and other medial materials. The small self-expandable stent used in this retrievable self-expandable covered stent disclosed herein may be manufactured using any suitable known techniques to make a metal sheet into a stent or to braid or weld metallic wires into a stent. The material used for the small distal self-expandable stent can be of any type including steel and nitinol. The small distal self-expandable stent can be closed-cell design.

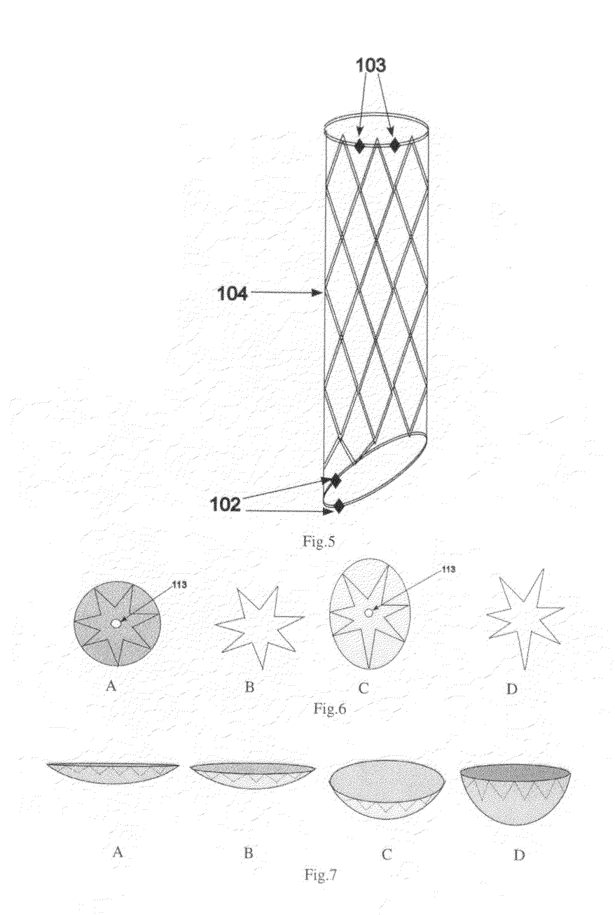

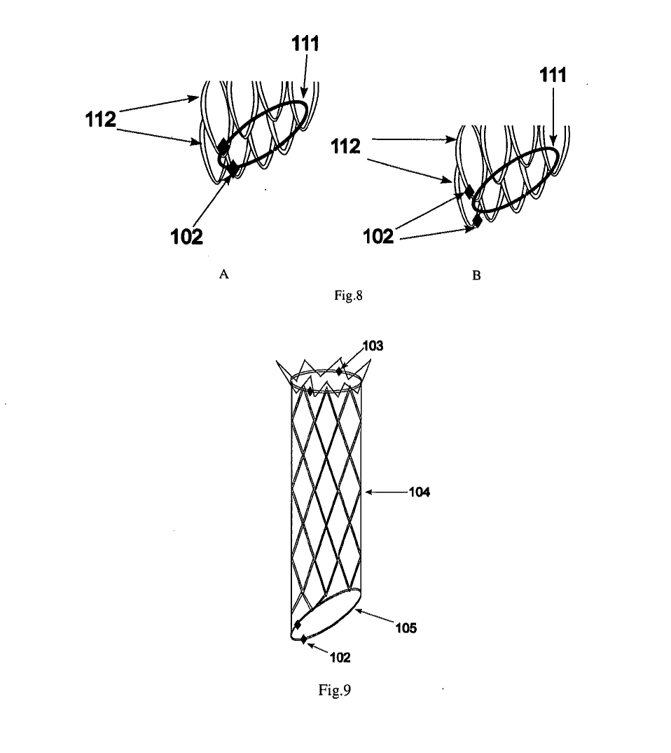

The lateral view of the distal small self-expandable covered stent (101) in full expansion is shown in FIG. 7 with different depth and diameter. As it can be seen from this figure, the distal small self-expandable covered stent is not completely on a plane when fully expanded. Rather, it is like an expanded umbrella in the expansion state. Thus, when the proximal supporting stent is retrieved and contracted, a contracting force will be exerted on the small distal self-expandable covered stent which will move and contract subsequently. Since the covering membrane is on the internal surface of the small distal stent, the membrane will not affect the contraction of the distal stent. The choosing of the distal small self-expandable covered stent is based on the size and shape of the bifurcation aneurysm neck and the bifurcation angle of the vessels. If the bifurcation angle is smaller, a deeper covered stent is chosen. If the bifurcation angle is bigger and flat, a shallower covered stent is chosen. In this way, the bifurcation aneurysm neck can be best occluded with no blood flow getting in.

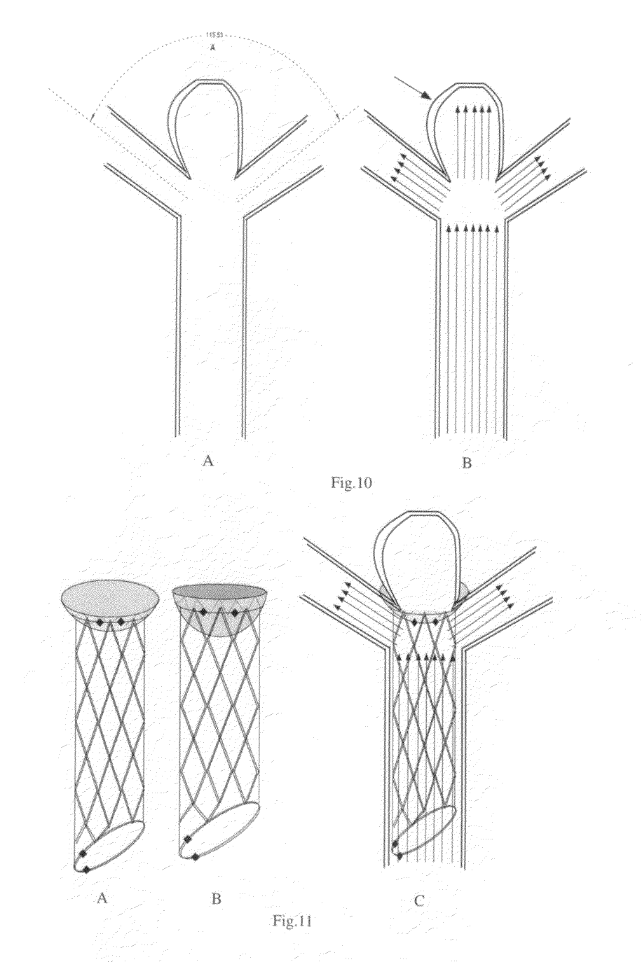

As shown in FIG. 8, the proximal retrieving structure (105) of the whole stent is consisted of a flexible circular metallic wire (111) and two markers (102) on it (A) or on the nearby stent struts (B, 112). The flexible circular wire (111) goes through the proximal stent struts (112) whose ends are not on the same horizontal level but on a slope. This particular structure enables the whole stent to be retrieved. When the flexible metallic wire (111) is pulled by a retrieving hook which hooks up the circular wire (111) between the two markers (102), the struts (112) will gather toward the hook. If the whole stent is fully expanded and in full contact with vascular walls, this action of pulling the flexible metallic wire (111) will enable the proximal part of the stent to contract and gradually detach from the vessel wall. If the flexible wire (111) is pulled into a catheter, the struts (112) near the hook will get into the catheter first, and struts farther away will follow into the catheter some time later. Because the end struts (112) of the proximal supporting stent (104) are not on the same horizontal level, the end struts of the whole stent will form a smaller profile to enter the catheter mouth easily. In this way, the struts of the proximal end of the stent will enter the catheter gradually, with some struts entering earlier while others later. Thus, the whole stent can be retrieved from the fully deployed state.

FIG. 9 demonstrates the proximal supporting stent connected to the distal small moveable self-expandable covered stent (101) without the covering membrane. 102 indicate the markers at the proximal end of the stent, 103 the markers at the distal end of the stent, 104 the proximal supporting self-expandable stent, and 105 the proximal retrieving structure for retrieving the stent. The manner the distal small self-expandable stent (101) is connected to the proximal supporting stent (104) is not from end to end but from end to stent trunk (middle part). This means that the distal end of the proximal supporting stent (104) is located in the middle of the struts of the small distal self-expandable stent so as to maintain the status of being movable of the small distal covered stent.

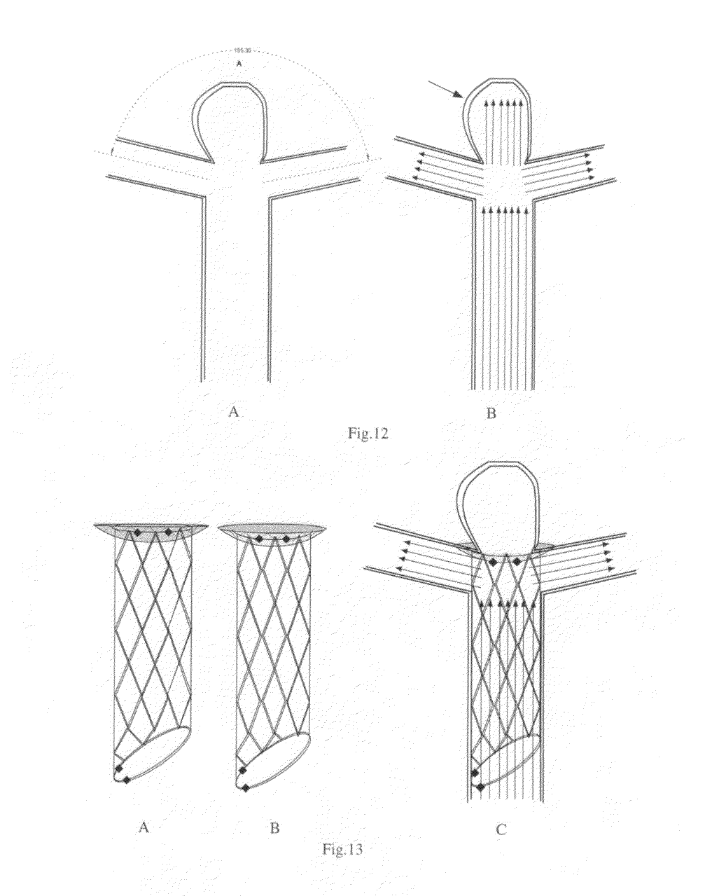

FIG. 10 and FIG. 11 illustrate the treatment of a bifurcation aneurysm with a smaller bifurcation angle A. The bifurcation angle is small, so a retrievable self-expandable covered stent with a deeper distal covered stent (101) is chosen so as to completely occlude the aneurysm neck. Deeper distal self-expandable covered stents are shown in FIGS. 11 A and B. After the deployment of the retrievable self-expandable covered stent at the vascular bifurcation, blood flow immediately stops getting into the aneurysm cavity (FIG. 11C) (comparing FIG. 10 B and FIG. 11 C).

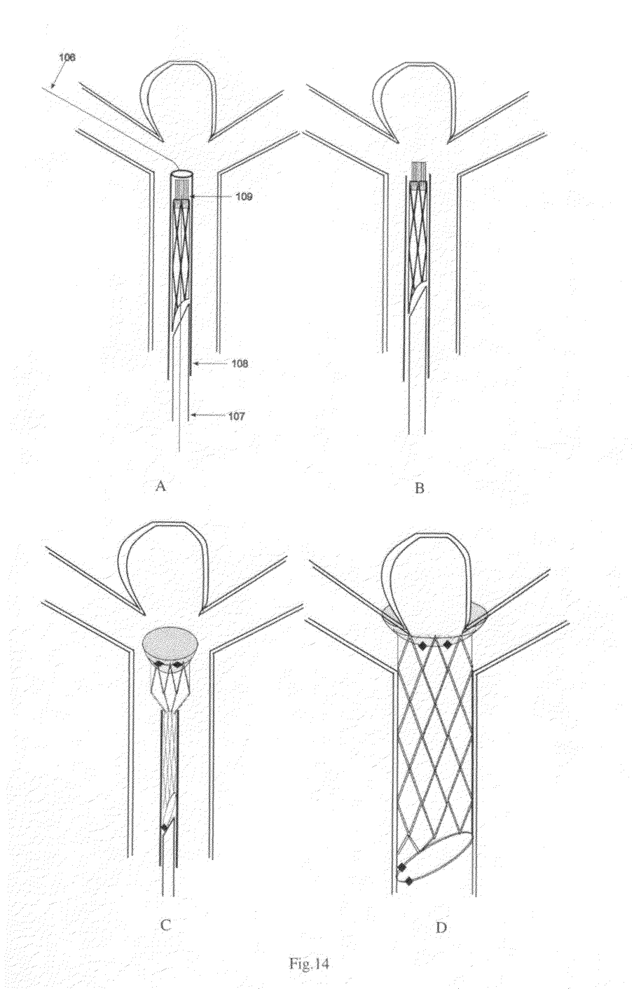

Referring to FIG. 12 and FIG. 13, the treatment of a bifurcation aneurysm with a bigger bifurcation angle is demonstrated. The bifurcation angle A is bigger, so a retrievable self-expandable covered stent with a shallower distal covered stent is chosen so as to completely occlude the aneurysm neck. Shallower distal self-expandable covered stents are shown in FIGS. 13 A and B. After the deployment of the retrievable self-expandable covered stent at the vascular bifurcation, blood flow immediately stops entering the aneurysm cavity (FIG. 13 C) (comparing FIG. 12 B and FIG. 13 C).

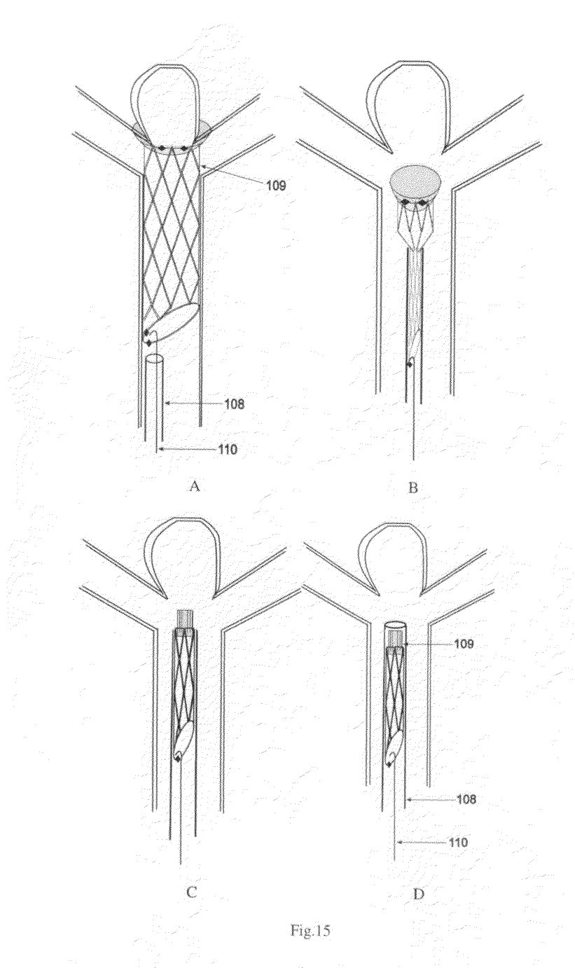

FIG. 14 demonstrates the delivery and deployment of the retrievable self-expandable covered stent. A guidewire (106) is advanced to one bifurcated branch, and then under the guidance of the guidewire (106), the catheter (108) with the whole retrievable covered stent (109) in it is navigated along the guidewire (106) to the appropriate position of the vascular bifurcation as shown in FIG. 14.A. After the removal of the guidewire (106), the pushing structure (107) is pushed forward to deploy the whole stent while the catheter (108) is gradually withdrawn (FIGS. 14.B and C). Finally, the catheter is completely withdrawn and the whole stent is deployed at the vascular bifurcation (FIG. 14.D). During the deployment process, the stent can be adjusted according to the position of the markers at both ends of the stent especially the markers at the distal end so that the aneurysm neck can be completely blocked by the distal moveable self-expandable covered stent. After deployment, inject some contrast material to see if the aneurysm neck is completely occluded or not. If not, the stent can be retrieved and readjusted before re-deployment.

FIG. 15 illustrates the process of retrieval of the whole stent. If the whole stent is not in the desired position and the aneurysm is not completely occluded, the whole stent can be retrieved and readjusted before redeployment. Insert a retrieving hook (110) through a catheter (108) to the proximal end of the stent (FIG. 15.A). The retrieving hook (110) is used to get hold of the proximal retrieving structure (105) of the stent at the most prominent struts between the two markers, and then slowly withdraw the retrieving hook (110) until the stent is partially or completely located within the catheter (FIG. 15.B-D). The whole stent may not be completely withdrawn into the catheter before stent adjustment, and the stent position can be adjusted during the step of either B or C. Redeploy the stent by using the pushing structure (107) so that the bifurcation aneurysm neck is completely occluded by the distal self-expandable covered stent.

What is claimed:

1. A retrievable covered stent which is used to treat bifurcation aneurysms comprising:
a. a smaller distal self-expandable covered stent composed of a membrane and a self-expandable stent;
b. a proximal self-expandable or balloon-expandable supporting stent for supporting and retaining the distal self-expandable covered stent;
c. a proximal retrieving structure connected to the self-expandable or balloon-expandable supporting stent for retrieving the whole stent.

2. The retrievable covered stent of claim 1, wherein the small distal self-expandable covered stent can expand like an umbrella to completely occlude the neck of an aneurysm in the vicinity of vascular bifurcations or trifurcations.

3. The retrievable covered stent of claim 2, wherein the graft material or membrane of the small distal self-expandable covered stent is stitched or glued to cover the internal surface of the small distal self-expandable stent.

4. The retrievable covered stent of claim 2, wherein the small distal self-expandable covered stent can be connected to the proximal supporting self-expandable or balloon-expandable stent.

5. The retrievable covered stent of claim 1, wherein the proximal supporting self-expandable or balloon-expandable stent has distal stent struts whose ends are on a horizontal level for connecting, supporting and retaining the small distal self-expandable covered stent.

6. The retrievable covered stent of claim 5, wherein the proximal ends of the stent struts of the proximal supporting self-expandable or balloon-expandable stent are on an incline plane or a slope rather than a horizontal level.

7. The retrievable covered stent of claim 5, wherein the proximal supporting self-expandable or balloon-expandable stent has some markers on the most prominent struts proximally for marking the retrieving position and the proximal end of the stent.

8. The retrievable covered stent of claim 5, wherein the proximal supporting self-expandable or balloon-expandable stent can be made of shape-memory alloy like nitinol or steel or any other materials, and can be expanded by itself or by a balloon.

9. The retrievable covered stent of claim 1, wherein the proximal retrieving structure can be used to retrieve the whole stent.

10. The retrievable covered stent of claim 9, wherein the proximal retrieving structure has a flexible circular metallic wire which connects or passes through the stent struts at the proximal end of the supporting self-expandable or balloon-expandable stent.

11. The retrievable covered stent of claim 9, wherein the flexible circular metallic wire of the proximal structure can be made of metal, alloy or any other strong materials for pulling and retrieving the whole stent.

12. A method for retrieving an endovascular stent or covered stent:
a. the retrievable stent or covered stent should have the stent struts at one end forming a slope or an incline plane rather than a horizontal or vertical plane even though the other end of the stent may be slope, horizontal or vertical;
b. when in the compressed state, the end of the struts forming a slope or an incline plane will constitute a smaller profile than the other segments of the stent;
c. a circular wire connects the stent struts at the end forming a slope for retrieval of the whole stent;
d. when the circular wire is captured by a hook and pulled toward the mouth of a catheter, the pulling of the wire will enable the proximal struts of the stent to compress and get into the catheter, and more distal struts will follow into the catheter until the whole stent enters the catheter gradually rather than all at the same time.
